SEQUENCE LISTING (7 sequences)

SEQ ID NO: 1 - protein, 10 aa, Artificial Sequence
  SYNTHESIZED.  ACTIVE FRAGMENT OF ISOLATE FROM OPOSSUM       SERUM. SEE US 5,576,297.
LKAMDPTPPL

SEQ ID NO: 2 - protein, 15 aa, Artificial Sequence
  SYNTHESIZED.  ACTIVE FRAGMENT OF ISOLATE FROM OPOSSUM       SERUM. SEE US 5,576,297.
LKAMDPTPPLWIKTE

SEQ ID NO: 3 - protein, 5 aa, Artificial Sequence
  SYNTHESIZED.  ACTIVE FRAGMENT OF ISOLATE FROM OPOSSUM       SERUM. SEE US 5,576,297.
LKAMD

SEQ ID NO: 4 - protein, 12 aa, Artificial Sequence
  Synthetic.  Corresponds to fragment 1-12 of 2 above.
LKAMDPTPPLWI

SEQ ID NO: 5 - protein, 11 aa, Artificial Sequence
  Synthetic.  Corresponds to fragment 1-11 of 2 above.
LKAMDPTPPLW

SEQ ID NO: 6 - protein, 9 aa, Artificial Sequence
  Synthetic.  Corresponds to fragment 1-9 of 2 above.
LKAMDPTPP

SEQ ID NO: 7 - protein, 8 aa, Artificial Sequence
  Synthetic.  Corresponds to fragment 1-8 of 2 above.
LKAMDPTP

(19) United States
(12) Patent Application Publication
(10) Pub. No.: US 2003/0157555 A1
(43) Pub. Date: Aug. 21, 2003

(54) Diagnosis and treatment for immunoglobulin E ( IgE) implicated disorders

(76) Inventors: Binie V. Lipps, Bellaire, TX (US); Frederick W. Lipps, Bellaire, TX (US)

Correspondence Address: John R. Casperson, PO Box 2174, Friendswood, TX, 77549, US

(21) Appl. No.: 10/047,945

(22) Filed: Jan. 14, 2002

(51) Int. Cl. G01N 33/53; G01N 33/537; G01N 33/543

(52) U.S. Cl. 435/7.1

(57) ABSTRACT

Human saliva is used as a non-invasive source instead of invasive blood serum plasma for detection and assay of endogenously present proteins; nerve growth factor (NGF), myoglobin, Insulin, adenosine deaminase (ADA), including immunoglobulin E (IgE). It was discovered that people having high levels of IgE, show high levels in comparison to the normal controls of NGF, myoglobin, insulin and ADA, disrupting the homeostasis for these proteins. Oral administration of a synthetic peptide LT-10 disclosed in U.S. Pat. No. 5,576,297 having sequence L K A M D P T P P L reduces IgE level in humans and bring other proteins into homeostasis, for example, NGF, myoglobin, insulin and ADA and possibly other proteins and cytokines. Composition of synthetic LT-10 is advocated as a treatment for IgE implicated disorders such as asthma, depression and various types of autoimmune diseases, such as erythematosus (SLE); Rheumatoid arthritis Sjogren's syndrome; Reiter's syndrome; Diabetes mellitus (insulin-dependent); Graves' disease; Addison's disease; Hodgkin's disease, etc.

### BACKGROUND OF THE INVENTION

### Field of the Invention

In one aspect, this invention relates to the introduction of use of saliva as a non-invasive source for detection and assay of endogenously present proteins, for example, nerve growth factor (NGF), myoglobin, Insulin, adenosine deaminase (ADA), and most importantly immunoglobulin E (IgE). In another aspect, the invention relates to the treatment of human disorders characterized by elevated IgE levels by the administration of a peptide to reduce the level.

IgE Implicated Disorders

A number of disorders and conditions are recognized by elevated levels of IgE. Human immunoglobulins are different types, such as IgG, IgA, IgM, IgD and IgE. IgG, IgA, and IgM are protective immunoglobulins. The role of IgD is not known. IgE is a minor component of total immunoglobulins and it is implicated in allergies, which in some cases manifests asthma. The presence of IgE in human serum was discovered in 1972 by Ishizaka K. and Ishizaka T. Normal adults have 0.2 to 1.0 mg % of IgE. Currently 20% of the US population has higher than the normal range of IgE and the percentage is increasing every year.

Allergic diseases are caused by adverse immune response to allergens. Allergen-sensitized patients produce high levels of IgE, which manifest vasodilation, increased vascular permeability, edema, smooth muscle contraction and mucus secretion, resulting allergic reactions. IgE is implicated in asthma because asthma people show high levels of IgE. Allergic reaction causes inflammation and edema accumulating mast cells (MC) at the sites, which remain active for producing IgE under different conditions. Exercise-induced allergies producing IgE is a common phenomenon among athletes. During emotional stress MC are activated to produce IgE stress.

NGF Implicated Conditions

There are publications stating that several inflammatory and autoimmune diseases are characterized by an altered concentration of circulating nerve growth factor (NGF). Enhanced NGF expression and production have been observed at the site of inflammation, where mast cells and activated immune cells accumulate. Levels of NGF in the serum of patients with inflammatory autoimmune disorders such as chronic arthritis (CA), Systemic scleroderma (SS), Systemic lupus erythematosus (SLE), and Multiple sclerosis (MS) were compared to their respective controls. It was reported that MS patients showed the highest level of NGF and CA patients showed the lowest in comparison to normal controls. Also, SLE and SS patients showed higher levels of NGF in comparison to normal controls. Whether the increase in NGF is directly responsible for inducing inflammation or just a consequence of the inflammatory process remains to be elucidated.

Numerous autoimmune diseases are recognized, for example, Systemic lupus erythematosus (SLE); Rheumatoid arthritis, Sjogren's syndrome; Reiter's syndrome; Diabetes mellitus (type II, not insulin-dependent); Graves' disease; Addison's disease Hodgkin's disease, etc. The etiology of, or the causative agents for, autoimmune diseases and for depression are not known. Therefore, they are referred as disorders rather than diseases.

Diabetes mellitus is a syndrome which affects many systems. It is a common condition, occurs in 3% human population. It occurs during middle age. Currently, the presence of elevated glucose in the blood is the only criterion upon which diagnosis of diabetes mellitus is based. There are no specific markers at this time. It is believed that insufficiency of metabolically active insulin may be implicated in development of long microvascular and neurological complications of diabetes. Recent research suggests the hypothesis that elements of the innate immune system, such as cytokines or the acute phase reactants that they stimulate, contribute to the development of type II diabetes and obesity. Furthermore, a recent publication by Lindsey et al. (2001) states that elevated levels of gamma globulins in blood can predict type II diabetes in Pima Indian population. These authors did not measure IgE levels.

Present Assay Techniques for Immunoglobulins and Other Proteins

For humans, immunoglobulins and other proteins are almost always assayed from serum. There is a reported data that IgG and IgA were assayed from saliva of BALB/c mice. Cytokines were assayed from human tears and it was found that elevated levels of inflammatory cytokines occurred in tears from persons with various ocular disease states. There is no published data reporting the use of saliva for assay of IgE and other proteins.

Desirability of Making IgE, NGF, Myoglobin, Insulin and ADA Determination from Saliva

The use of saliva for assaying endogenous proteins has several advantages over the current practice and use of serum. Saliva collection is non invasive, while blood collection for serum is invasive. Saliva collected in a tube can be centrifuged immediately to get rid of cells, while blood requires clotting time before it can be centrifuged to separate serum. Saliva proteins can be assayed by a simple antigen antibody Enzyme-linked Immunosorbent (ELISA) test, whereas an assay of proteins from serum requires sandwich type ELISA, which is more complicated. It requires more time and reagents. In case of saliva the controls for ELISA have negligible background, whereas for serum the background noise has to be monitored carefully. Therefore, considering the above points the use of saliva as a source to assay proteins can be done by a simple ELISA test with reproducible results.

Reducing IgE Level

A reduction in IgE should lead to reduction in IgE-mediated symptoms and therefore, can control allergic/rhinitis and asthma. A reagent to reduce elevated IgE level in humans would be desirable. It has been proposed to use monoclonal antibodies against IgE (mono-anti-IgE) to reduce IgE level in asthma patients. However, a large protein molecule of mono-anti-IgE would be effective only by injection. Small molecule having low molecular weight can be given orally would be very desirable.

### OBJECTS OF THE INVENTION

An object of the invention is to use saliva in place of currently practiced invasive blood serum collection for assaying endogenously present proteins, such as IgE, NGF, Myoglobin, Insulin and ADA.

Our research further revealed that IgE is implicated in (1) Type II diabetes (2) Depression (3) various types of Autoimmune diseases and (4) Asthma. It was revealed that the level of IgE in patients of these disorders is several times higher than the control normal individuals. High level of IgE is found in allergy/asthma patients. This invention provides the assay of IgE in saliva of patients afflicted with Asthma, Diabetes, Depression and various kinds of autoimmune diseases. This information can be used in diagnosis and in treatment.

We also found that high levels of IgE caused disruption in the homeostasis of endogenously present other proteins such as nerve growth factor, myoglobin, insulin and Adenosine deaminase. We believe that such disruption in homeostasis for NGF, myoglobin, insulin and ADA may be manifesting the symptoms for these disorders. For example, a high level of myoglobin may be implicated with a heart problem; a high level of insulin may indicate involvement of pancrease. It is known that a high level of ADA is due to asthma and involvement of lungs.

Currently, there is no treatment to lower the level of IgE, although 20% population shows high level of IgE and there is yearly increasing percentage. Genentech Corp. has proposed a monoclonal antibody treatment for asthma. The costly drug consisting of monoclonal antibody is given by several injections in milligram amounts to lower IgE level to prevent asthma attacks only. Administration of monoclonal antibody is a passive process of immunization. The life period of such passive antibody is a limited short period. Furthermore, excess monoclonal antibody, not bound to free IgE, is liable to generate anti-anti-IgE or anti-idiotypic antibody which can interfere with treatment.

Another object of the present invention is to provide a novel therapeutic for the treatment of IgE implicated disorders in people having high levels of IgE, for example, people having Asthma, Diabetes, Depression and various kinds of autoimmune diseases. We demonstrated that in humans oral administration of a synthetic Lethal Toxin Neutralizing Factor (LTNF) designated LT-10 lowers IgE level. We further demonstrated that by lowering the IgE level, other proteins such as NGF, myoglobin, insulin and ADA returned to their normal homeostasis.

The synthetic LTNF is described in U.S. Pat. No. 5,576,297 (1996) “Embodiments of Natural and Synthetic Lethal Toxin Neutralizing Factors (LTNFs) and their utility as treatment for Envenomation” and U.S. Pat. No. 5,744,449 (1998) “Lethal Toxin Neutralizing Factors.” The disclosures of these patents are incorporated by reference herein. After identifying the active domain of natural LTNF, synthetic LTNF designated as LT-10 was made using ten amino acids having a sequence from the N-terminal of L K A M D P T P P L (Leu Lys Ala Met Asp Pro Thr Pro Pro Leu—SEQ ID NO 1). Another version designated LT-15 consisting of 15 amino acids and a sequence from the N-terminal of L K A M D P T P P L W I K T E (Leu Lys Ala Met Asp Pro Thr Pro Pro Leu Trp Ile Lys Thr Glu—SEQ ID NO 2); and another version designated LT-5 consisting of 5 amino acids and a sequence from the N-terminal of L K A M D (Leu Lys Ala Met Asp—SEQ ID NO 3) were also made. Al three versions; LT-15, LT-10 and LT-5 have similar biological activity and are useful in this invention as are the peptides of intermediate length. For convenience, the invention is largely described hereinafter with reference to LT-10, although the invention should not be construed as being so limited.

The proposed treatment with LT-10 to lower the concentration of IgE has several advantages over the contemplated use of monoclonal antibodies against IgE (Mono anti-IgE). LT-10 is a synthetic peptide made of 10 amino acids, which can be made in abundance and very chiefly. Mono anti-IgE is a big protein molecule and the cost can be $ 3,000 to 5,000 per mg. LT-10 can be given orally under the tongue. Mono anti-IgE must be given by injection only. Being a large molecule, it will not be absorbed by oral administration. Both LT-10 and Mono anti-IgE neutralize the circulating IgE and lower the IgE level. Excess LT-10 in the system will not do any harm. However, excess of Mono anti-IgE unused will start making antibodies. These anti idiotypic antibodies or anti-anti Mono IgE, which is a copy of IgE, will interfere with treatment. We propose LT-10 treatment should be continuous in order to maintain IgE level to normal state. Because, IgE level is known to rise under environmental, emotional stress and exercise etc., mono anti-IgE treatment can not be given continuously due route of delivery and expense etc.

### SUMMARY OF THE INVENTION

We have discovered that elevated IgE characterizes disorders other than asthma.

We have discovered that IgE levels can be determined from saliva.

We have found that IgE levels can be reduced by treatment by LT-10 and related peptides.

We have found that a reduction in IgE levels brings concommitant reduction in certain other serum proteins which are disease and/or risk indicators.

We have found that LT-10 and related peptides are effective for this purpose when given orally.

### BRIEF DESCRIPTION OF THE DRAWINGS

FIG. 1 graphically illustrates experimental results obtained from certain of the examples.

### DETAILED DESCRIPTION OF THE INVENTION

In one embodiment of the invention, there is provided a method for assaying human endogenous proteins from saliva. A saliva sample is obtained and an ELISA assay performed on the sample employing an anti-serum which is specific for the protein of interest.

Useful information is obtained by analyzing for at least one of IgE, NGF, Insulin, Myoglobin and ADA. The ELISA is performed with anti-IgE, anti-NGF, anti-Insulin, anti-Myoglobin, and anti-ADA, as applicable.

Elevated levels of serum proteins selected from the group consisting of IgE, NGF, Insulin, Myoglobin and ADA can be reduced by administering to said human exhibiting such level an effective amount of a peptide containing at least the first four amino acids from the N-terminal of the sequence Leu Lys Ala Met Asp Pro Thr Pro Pro Leu Trp Ile Lys Thr Glu. Preferably, the peptide contains the sequence of at the least first four amino acids beginning at its N-terminal and has no more than 20 amino acids total, and more preferably has in the range of from five to fifteen amino acids total. Most preferably, the peptide has from eight to 12 amino acids total and is selected from the group of peptides

Leu Lys Ala Met Asp Pro Thr Pro Pro Leu Trp Ile (SEQ ID NO 4),

Leu Lys Ala Met Asp Pro Thr Pro Pro Leu Trp (SEQ ID NO 5),

Leu Lys Ala Met Asp Pro Thr Pro Pro Leu (SEQ ID NO 1),

Leu Lys Ala Met Asp Pro Thr Pro Pro (SEQ ID NO 7), and

Leu Lys Ala Met Asp Pro Thr Pro (SEQ ID NO 8).

By using peptides as described above, the peptide can be and preferably is orally administered and serum IgE level is reduced.

Generally speaking, in the range of from about 0.02 to about 200 milligrams of the peptide is orally administered on a daily basis, usually in the range of from about 0.2 to about 20 milligrams on a daily basis. Oral administration of an amount of the 10 amino acid peptide within the range of 0.2 to 5 milligrams daily has been demonstrated to markedly influence blood protein levels, and an amount in the range of 0.5 to about 2 milligrams daily has been tested with good results. Usually, the peptide is administered to humans having an elevated serum IgE level, as compared to norms. Often, a patient having an elevated IgE level will also have an elevated NGF, Insulin, Myoglobin and/or ADA serum level.

The peptide is believed effective to treat conditions selected from the group consisting of Asthma, Diabetes, Depression and Autoimmune Disease. Typical autoimmune diseases are selected from the group consisting of erythematosus (SLE), Rheumatoid arthritis, Sjogren's syndrome, Reiter's syndrome, Graves' disease, Addison's disease, and Hodgkin's disease.

### EXPERIMENTAL

Following experiments were performed.

Experiment 1: The pool of several human salivas was split into two parts. To one part equal volume of PBS was added and to the second part equal volume containing 1 mg/ml of LT-10 was added. The mixtures were incubated at 37° C. for one hour. IgE levels were assayed in both mixtures by usual ELISA test using anti-IgE. It was revealed that IgE level was much reduced in the mixture of saliva and LT-10, in comparison to the mixture of saliva and PBS. This shows the binding of LT-10 to IgE in saliva, the bound IgE is not detected by anti-IgE by ELISA test.

Experiment 2: I placed one ml of water in my mouth and kept it for 15 minutes, after which the mixture with saliva and water was collected. Likewise I placed one ml of LT-10 containing 1 mg/ml and the mixture of saliva and LT-10 was collected. IgE levels were assayed in both mixtures by usual ELISA test. It was revealed that IgE level was much reduced in the mixture of saliva and LT-10, in comparison to the mixture of saliva and water. This shows that the binding of LT-10 to IgE in saliva in mouth.

So far anti-IgE treatment is advocated only for allergic rhinitis and asthma. After discovering the high levels of IgE implicated for other than asthma disorders, we advocate LT-10 treatment for the disorders where IgE levels are high, those are:

(1) Type II diabetes (2) Depression (3) various types of Autoimmune disorders and (4) Asthma.

Currently, diabetes, depression and autoimmune diseases are treated with various drugs. For example, diabetes treated by insulin injections, and depression with anti depression drugs like Prozac. Autoimmune disorders are treated with immuno-suppressive drugs. We obtained saliva from the people who are undergoing treatment for their respective disorders for years. Our results emphasize that in spite of the conventional treatment, IgE levels remained very high causing disruption in homeostasis of other proteins. The elevated levels of NGF, myoglobin, insulin, and ADA, are measured in saliva of the people having high concentration of IgE indicating damage of various organs.

LT-10 treatment lowers the IgE level and the levels of other measured proteins. We believe that LT-10 treatment is ideal for these diseases and LT-10 has no observable side effects.

Human Saliva: Saliva from individual was collected in a centrifuge tube. Collected saliva was centrifuged and the supernatant was separated. Protein concentration of the saliva was measured by spectrophotometer. The protein content for saliva was adjusted to 200 μg/ml and stored frozen from which it was diluted in carbonate-bicarbonate buffer pH 9.4 to give the concentration 10 μg/ml for ELISA tests.

Following antisera were used to assay IgE, NGF, Insulin, Myoglobin and ADA. Anti-IgE, and anti-NGF were made in house, by immunizing rabbits. Anti-Myoglobin made in rabbits was purchased from OEM concepts; Anti-insulin made in pig was purchased from Sigma-Aldrich Co. Anti-ADA is not available commercially was made in house by immunizing BALB/c mice.

Enzyme-Linked Immunosorbent Assay (ELISA) for Human Saliva:

ELISA tests were performed in 96 well micro-plate. The wells of the plate were coated with saliva at 10 μg/ml concentration in carbonate-bicarbonate buffer pH 9.4, each well receiving 100 μl. After overnight incubation at room temperature the plate was washed three times with 0.05 phosphate buffered saline (PBS). Anti-IgE diluted in 3% gelatin from 1:100 to 1:2187 was added to three wells for each dilution. Similar procedure was followed for assaying NGF, myoglobin insulin and ADA by using respective anti-sera; such as anti-NGF, anti-myoglobin; anti-insulin and anti-ADA. Antigen-antibody reaction was carried at 37° C. for 1.5 hours. After which the plate was washed and was reacted with horseradish peroxidase conjugated with IgG. Rabbit horseradish peroxidase was reacted for rabbit anti-IgE and anti-NGF; pig peroxidase for pig anti-insulin and mouse peroxidase for mouse anti-ADA.

Assays of endogenously present proteins, IgE, NGF, Myoglobin, Insulin and ADA in human saliva are compared with the normal control counterparts. The results are presented in tables 1 and 2. The ELISA titers for IgE, NGF, myoglobin, Insulin and ADA were divided by a normal ELISA titer, to give the normalized reading.

TABLE 1
High Level of IgE corresponds to high levels of NGF and Myoglobin in
human saliva.

| Speci- | | | IgE/ | | NGF/ | | Myo/ |
|---|---|---|---|---|---|---|---|
| men | Status | IgE | Norm | NGF | Norm | Myo | Norm |
| Pool of | Normal | 12150 | 1.00 | 1200 | 1.00 | 1800 | 1.00 |
| 6 | | | | | | | |
| Pool of | Marginal | 32400 | 2.67 | 1800 | 1.50 | 2700 | 1.50 |
| 2 | | | | | | | |
| Pool of | Diabetes | 145800 | 12.00 | 5400 | 4.50 | 3600 | 2.00 |
| 2 | | | | | | | |
| JC | Diabetes | 145800 | 12.00 | 24300 | 20.25 | 10800 | 6.00 |
| TF | Asthma | 145800 | 12.00 | 5400 | 4.50 | 5400 | 3.00 |
| WK | Depres- | 218700 | 18.00 | 24300 | 20.25 | 16200 | 9.00 |
| | sion | | | | | | |
| WC | Normal | 16200 | 1.33 | 2700 | 2.25 | 1800 | 1.00 |
| RC | Auto-imm | 72900 | 6.00 | 5400 | 4.50 | 3600 | 2.00 |
| BS | Auto-imm | 218700 | 18.00 | 8100 | 6.75 | 10800 | 6.00 |
| RG | Auto-imm | 48600 | 4.00 | 1800 | 1.50 | 1800 | 1.00 |
| AA | Auto-imm | 72900 | 6.00 | 2700 | 2.25 | 3600 | 2.00 |
| SG | Auto-imm | 72900 | 6.00 | 8100 | 6.75 | 5400 | 3.00 |
| RC | Auto-imm | 437400 | 36.00 | 24300 | 20.25 | 32400 | 18.00 |
| JC | Auto-imm | 437400 | 36.00 | 24300 | 20.25 | 32400 | 18.00 |
| VA | Auto-imm | 48600 | 4.00 | 2700 | 2.25 | 1800 | 1.00 |
| GA | Auto-imm | 437400 | 36.00 | 16200 | 13.50 | 32400 | 18.00 |
| NG | Auto-imm | 48600 | 4.00 | 2700 | 2.25 | 5400 | 3.00 |
| Nor- | | 12150 | | 1200 | | 1800 | |
| mal | | | | | | | |

Results of Table 1 show that:

(1) IgE levels are higher than normal in saliva from diabetes, asthma, depression and various types of autoimmune disorders. IgE level varied from 2.67 times as in the marginal normal people to 36 times as in autoimmune disorder patients in comparison to normal counterpart.

(2) Patients showing high levels of IgE showed high levels of NGF. NGF levels varied from 4.5 times in diabetes to 20.25 times in depression and autoimmune disorders.

(3) Patients showing high levels of IgE showed high levels of myoglobin. Myoglobin levels varied from 3.0 times in asthma patient to 18.0 times in autoimmune disorders.

TABLE 2
High Level of IgE corresponds to high levels of Insulin and ADA in
human saliva.

| Speci- | | | IgE/ | | Ins/ | | ADA/ |
|---|---|---|---|---|---|---|---|
| men | Status | IgE | Norm | Insulin | Norm | ADA | Norm |
| Pool of | Normal | 12150 | 1.00 | 450 | 1.00 | 600 | 1.00 |
| 6 | | | | | | | |
| Pool of | Marginal | 32400 | 2.67 | 600 | 1.33 | 900 | 1.50 |
| 2 | | | | | | | |
| Pool of | Diabetes | 145800 | 12.00 | 1800 | 4.00 | 1800 | 3.00 |
| 2 | | | | | | | |
| JC | Diabetes | 145800 | 12.00 | 1800 | 4.00 | 1800 | 3.00 |
| TF | Asthma | 145800 | 12.00 | 2700 | 6.00 | 8100 | 13.5 |
| WK | Depres- | 218700 | 18.00 | 1800 | 4.00 | 2700 | 4.50 |
| | sion | | | | | | |
| WC | Normal | 16200 | 1.33 | 300 | 0.67 | 600 | 1.00 |
| RC | Auto-imm | 72900 | 6.00 | 450 | 1.00 | 2700 | 4.50 |
| BS | Auto-imm | 218700 | 18.00 | 2700 | 6.00 | 2700 | 4.50 |
| RG | Auto-imm | 48600 | 4.00 | 450 | 1.00 | 450 | 0.75 |
| AA | Auto-imm | 72900 | 6.00 | 450 | 1.00 | 450 | 0.75 |
| SG | Auto-imm | 72900 | 6.00 | 2700 | 6.00 | 450 | 0.75 |
| RC | Auto-imm | 437400 | 36.00 | 2700 | 6.00 | 2700 | 4.50 |
| JC | Auto-imm | 437400 | 36.00 | 2700 | 6.00 | 1800 | 3.00 |
| VA | Auto-imm | 48600 | 4.00 | 900 | 2.00 | 900 | 1.50 |
| GA | Auto-imm | 437400 | 36.00 | 1800 | 4.00 | 1800 | 3.00 |
| NG | Auto-imm | 48600 | 4.00 | 900 | 2.00 | 900 | 1.50 |
| Nor- | | 12150 | | 450 | | 600 | |
| mal | | | | | | | |

Results of Table 2 show

(1) IgE levels are higher than normal in saliva from diabetes, asthma, depression and various types of autoimmune disorders. IgE level varied from 2.67 times as in the marginal normal people to 36 times as in autoimmune disorder patients in comparison to normal counterpart.

(2) Patients showing high levels of IgE showed high levels of Insulin. Insulin levels varied from 4.0 times in diabetes patient to 6.0 times in autoimmune disorders.

(3) Patients showing high levels of IgE showed high levels of ADA especially in asthma patient, 13.5 times greater than normal. Some autoimmune patients showed lower level of ADA in comparison to normal people. Thus ADA level varied from 0.7 to 6 times.

Collectively, the results of Tables 1 and 2 clearly show that the elevated level of IgE is the culprit—causing numerous types of disorders. The elevated level caused increased levels for other proteins such as NGF, Myoglobin, insulin and in case of asthma ADA.

Personal Example from the Inventor Binie Lipps:

On my annual medical check, I was diagnosed to be diabetes based on the high level of glucose in blood, the only available test for diagnosis. I did not have discomfort or symptoms. I took Glucotrol treatment for two months as was prescribed by the doctor. After two months of Glucotrol treatment and sugar-free diet, the blood glucose level came down but remained high. I often used to get allergic reactions. Therefore, I realized that high glucose in blood may be related to allergic reaction. In the meantime, I discovered that IgE can be assayed from saliva. Before that, an assay of IgE was possible only from an invasive procedure to obtain a serum specimen. I also discovered that the endogenously present other proteins, NGF, Myoglobin, Insulin and ADA can be assayed from saliva by ELISA test.

After the discovery that IgE could be assayed from saliva, the following experiments were performed. Fasting saliva collected and glucose level measured for seven days for each experiment. Sugar free diet was observed during all experiments. In addition to IgE, NGF, Myoglobin, Insulin and ADA were assayed in saliva. After completion of an experiment two day waiting period was allowed before starting the next experiment.

Experiment #1: No treatment.

Experiment #2: Glucotrol treatment, 10 mgs in the morning and 5 mgs in the evening.

Experiment #3: LT-10 treatment 2 mgs/day, 1 mg in the morning and 1 mg in the evening

Experiment #4: Combination of LT-10, 2 mgs/day and 15 mgs/day Glucotrol.

The results of these experiments are shown in tables 3 to 7.

TABLE 3
Blood Glucose level in mgs:

| | Treatment | | | |
|---|---|---|---|---|
| | None | Gluco | LT-10 | Combination |
| Day | Expt #1 | Expt #2 | Expt #3 | Expt #4 |
| 1 | 305 | 183 | 132 | 137 |
| 2 | 244 | 183 | 124 | 145 |
| 3 | 144 | 209 | 116 | 140 |
| 4 | 186 | 199 | 123 | 142 |
| 5 | 203 | 218 | 151 | 150 |
| 6 | 191 | 208 | 183 | 158 |
| 7 | 116 | 214 | 155 | 150 |

The results show that the glucose level remained variable in all four experiments. In expt #1 sugar level fluctuated from 116 to 301. In expt #2 glucose level fluctuated from 183 to 214, with Glucotrol treatment, did not make appreciable difference for glucose. However, in experiment# 3 and in expt #4 the glucose levels remained lower in comparison to expt #1 and # 2. Fluctuation in expt #3 was 116 to 183 and in expt #4 137 to 158. Glucotrol treatment may be lowering glucose level as in exp#2. However, it is not Glucotrol but LT-10 lowered the glucose level as in expt #3 and #4.

TABLE 4
IgE levels in saliva:

| | Treatment | | | |
|---|---|---|---|---|
| | None | Gluco | LT-10 | Combi |
| Day | Expt #1 | Expt #2 | Expt #3 | Expt #4 |
| 1 | 145800 | 145800 | 145800 | 145800 |
| 2 | 148600 | 148600 | 72900 | 145800 |
| 3 | 148600 | 145800 | 72900 | 145800 |
| 4 | 148600 | 148600 | 72900 | 72900 |
| 5 | 145800 | 148600 | 72900 | 72900 |
| 6 | 145800 | 145800 | 72900 | 48600 |
| 7 | 145800 | 145800 | 48600 | 24300 |

Normal 16200

IgE levels remained high in expt # 1 and 2 with no treatment or Glucotrol treatment. LT-10 treatment alone for seven days as in expt #3 or in combination with Glucotrol as in exp#4 lowered the IgE levels almost reaching to normal. Results clearly indicate that Glucotrol treatment does not contribute in lowering IgE levels. It is the LT-10 treatment which causes the lowering of IgE.

TABLE 5
NGF levels in saliva:

| | Treatment | | | |
|---|---|---|---|---|
| | None | Gluco | LT-10 | Combi |
| Day | Expt #1 | Expt #2 | Expt #3 | Expt #4 |
| 1 | 2700 | 2700 | 2700 | 2700 |
| 2 | 2700 | 8100 | 2700 | 3600 |
| 3 | 2700 | 5400 | 2700 | 3600 |
| 4 | 2700 | 5400 | 1800 | 3600 |
| 5 | 2700 | 2700 | 1800 | 2700 |
| 6 | 5400 | 2700 | 1800 | 2700 |
| 7 | 5400 | 5400 | 1800 | 2700 |

Normal 1200

NGF levels remained high in expt # 1 and 2 with no treatment or Glucotrol treatment. LT-10 treatment alone for seven days as in expt#3 lowered the NGF levels almost to normal. It seems that as in expt #2 with Glucotrol alone and in expt #4 the combination of LT-10 and Glucotrol caused elevation in NGF. Results clearly indicate that Glucotrol treatment does not contribute in lowering NGF levels. On the contrary, Glucotrol perhaps increases NGF levels. LT-10 treatment causes the lowering of NGF to bring normal homeostasis.

TABLE 6
Insulin levels in saliva:

| | Treatment | | | |
|---|---|---|---|---|
| | None | Gluco | LT-10 | Combi |
| Day | Expt #1 | Expt #2 | Expt #3 | Expt #4 |
| 1 | 2700 | 1200 | 2700 | 2700 |
| 2 | 2700 | 800 | 1800 | 1800 |
| 3 | 1800 | 800 | 1800 | 2700 |
| 4 | 1200 | 900 | 1800 | 2700 |
| 5 | 1800 | 750 | 1800 | 1800 |
| 6 | 1800 | 800 | 1800 | 900 |
| 7 | 2700 | 900 | 900 | 900 |

Normal 600

Insulin levels remained high in expts# 1 and 2 with no treatment or Glucotrol treatment. LT-10 treatment alone for seven days as in expt #3 or in combination with Glucotrol as in expt #4 lowered the Insulin levels to almost normal. Results indicate that perhaps Glucotrol treatment contributes in lowering Insulin levels as expts #2 and 4.

TABLE 7
Myoglobin levels in saliva:

| | Treatment | | | |
|---|---|---|---|---|
| | None | Gluco | LT-10 | Combi |
| Day | Expt #1 | Expt #2 | Expt #3 | Expt #4 |
| 1 | 1800 | 1800 | 1800 | 2700 |
| 2 | 1800 | 3600 | 1800 | 3600 |
| 3 | 3600 | 3600 | 2700 | 3600 |
| 4 | 3600 | 2700 | 1800 | 3600 |
| 5 | 1800 | 2700 | 1800 | 2700 |
| 6 | 2700 | 2700 | 1800 | 2700 |
| 7 | 1800 | 3500 | 1800 | 2700 |

Normal 1800

Myoglobin levels remained high in expts #1 and 2 with no treatment or Glucotrol treatment. LT-10 treatment alone for seven days as in expt #3 lowered the myoglobin levels to almost normal. Results indicate that perhaps Glucotrol treatment contributes in increasing myoglobin levels as seen in expts #2 and 4. This side effect of Glucotrol treatment may be implicated to heart trouble.

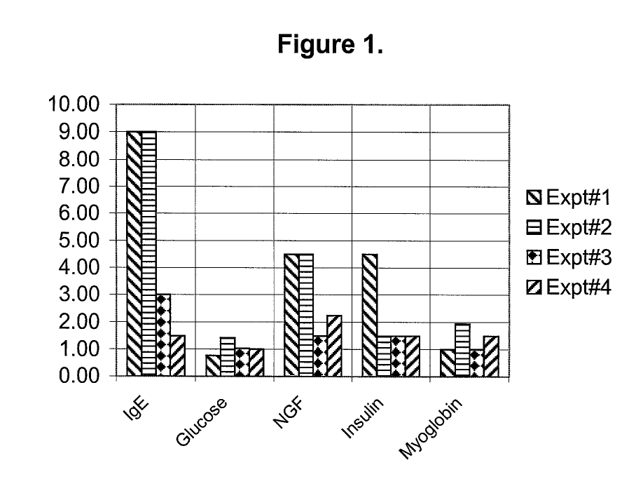

In FIG. 1, Expt#1 is no treatment, Expt#2 is Glucotrol treatment, Expt#3 is LT-10 treatment and Expt#4 is Glucotrol+LT-10. The levels of IgE, Glucose, NGF, Insulin and myoglobin are expressed as times the normal level of the respective protein.

The results of the four experiments at the completion point which is the end of seven days are graphically illustrated in FIG. 1:

1. IgE level remained high in expts#1 and #2. Lowered by LT-10 treatment as in expt#3 and with combination treatment.

2. Glucose level responded variously in the experiments.

3. NGF level remained high at the end of expts #1 and #2. LT-10 alone or in combination with Glucotrol as in expts #3 and #4 lowered the level NGF.

4. Insulin level decreased in all three expts #2, 3, 4.

5. Glucotrol treatment alone or in combination with LT-10 increased the level of myoglobin.

We claim:

1. The invention relates to the discovery of the presence of endogenous proteins; nerve growth factor (NGF), myoglobin, Insulin, adenosine deaminase (ADA), including immunoglobulin E (IgE) in samples of human saliva.

2. This invention relates to the introduction of use of saliva as a non-invasive source for detection and assay of endogenously present proteins; NGF, myoglobin, Insulin, ADA, including IgE.

3. The invention discovered that people having high levels of IgE show higher levels in comparison to controls, of NGF, myoglobin, insulin and in asthma patients high level of ADA, disrupting the homeostasis for these proteins.

4. Composition of synthetic LT-10 is advocated as a treatment for IgE implicated disorders. Synthetic LT-10 consists often amino acids: Leu Lys Ala Met Asp Pro Thr Pro Pro Leu.

5. Composition of synthetic LT-10 as a treatment for having high level of IgE, causes decrease in level of IgE. Decrease in IgE level brings the homeostasis of other proteins to normal state which improves the symptoms in the afflicted people for asthma, depression, diabetes and various types of autoimmune diseases, such as erythematosus (SLE); Rheumatoid arthritis Sjogren's syndrome; Reiter's syndrome; Diabetes mellitus (insulin-dependent); Graves' disease; Addison's disease Hodgkin's disease etc.

6. People having elevated level of IgE also show high level of NGF may be prone to the symptoms of neuropathy. Likewise high level of myoglobin may cause heart problems. Elevated level of insulin shows pancreases is affected. LT-10 treatment decreases the level of IgE which brings the homeostasis for NGF, myoglobin, insulin, ADA and may be other proteins and cytokines to normal status.

7. A method for assaying a human endogenous protein of interest, said method comprising
obtaining a saliva sample from a human, and
performing an ELISA assay on such saliva sample employing an anti-serum which is specific for the protein of interest.

8. A method as in claim 7 wherein the analysis is performed for at least one protein selected from the group consisting of IgE, NGF, Insulin, Myoglobin and ADA and the ELISA is performed with anti-IgE, anti-NGF, anti-Insulin, anti-Myoglobin, and anti-ADA.

9. A method for reducing serum proteins selected from the group consisting of IgE, NGF, Insulin, Myoglobin and ADA in a human, comprising
administering to said human an effective amount of a peptide containing at least the first four amino acids from the N-terminal of the sequence
Leu Lys Ala Met Asp Pro Thr Pro Pro Leu Trp Ile Lys Thr Glu
to reduce at least one serum level of IgE, NGF, insulin, myoglobin and ADA in said human.

10. A method as in claim 9 wherein the peptide contains the sequence of at the least first four amino acids beginning at its N-terminal and has no more than 20 amino acids total.

11. A method as in claim 10 wherein the peptide is orally administered and serum IgE level is reduced.

12. A method as in claim 10 wherein the range of from about 0.02 to about 200 milligrams of the peptide are orally administered on a daily basis.

13. A method as in claim 10 wherein in the range of from about 0.2 to about 20 milligrams of the peptide are orally administered on a daily basis and the peptide is selected from the group consisting of
Leu Lys Ala Met Asp Pro Thr Pro Pro Leu Trp Ile,
Leu Lys Ala Met Asp Pro Thr Pro Pro Leu Trp,
Leu Lys Ala Met Asp Pro Thr Pro Pro Leu,
Leu Lys Ala Met Asp Pro Thr Pro Pro, and
Leu Lys Ala Met Asp Pro Thr Pro.

14. A method as in claim 10 wherein said human has an elevated serum IgE level.

15. A method as in claim 14 wherein said human further has an elevated NGF, Insulin, Myoglobin and/or ADA serum level.

16. A method as in claim 14 further comprising assaying a saliva IgE level in said human.

17. A method as in claim 14 comprising
diagnosing a condition selected from the group consisting of
Asthma, Diabetes, Depression and Autoimmune Disease
in said human to which said peptide is to be administered.

18. A method as in claim 17 wherein the autoimmune disease is selected from the group consisting of erythematosus (SLE), Rheumatoid arthritis, Sjogren's syndrome, Reiter's syndrome, Graves' disease, Addison's disease, and Hodgkin's disease.
